(19) United States
(12) Patent Application Publication
(10) Pub. No.: US 2024/0091412 A1
(43) Pub. Date: Mar. 21, 2024

(54) CURABLE COMPOUNDS AND FORMULATIONS FOR BIOMEDICAL APPLICATIONS

(71) Applicant: 3D SYSTEMS, INC., Rock Hill, SC (US)

(72) Inventors: Pingyong Xu, Valencia, CA (US); Michael Brady, San Diego, CA (US); Jeff Banning, North Wilsonville, OR (US); Sarah Patty, Beaverton, OR (US); Peter Scott Turner, Venice, CA (US); Monica Keilsohn, San Diego, CA (US)

(21) Appl. No.: 17/949,484

(22) Filed: Sep. 21, 2022

Related U.S. Application Data
(60) Provisional application No. 63/246,604, filed on Sep. 21, 2021.

(51) Int. Cl. A61L 27/52; A61L 27/18; A61L 27/26; B33Y 80/00; C08L 71/02

(52) U.S. Cl. CPC A61L 27/52; A61L 27/18; A61L 27/26; B33Y 80/00; C08L 71/02; A61L 2400/16

(57) ABSTRACT

Curable compounds, hydrogels, build materials, and methods of 3D printing are described herein. In some embodiments, a build material for 3D printing described herein comprises one or more compounds having the structure(s) of Formula (I) and/or Formula (II) herein. Such a build material may also comprise an additional acrylate component and water.

### CROSS REFERENCE TO RELATED APPLICATIONS

This application claims priority pursuant to 35 U.S.C. § 119 to U.S. Provisional Patent Application No. 63/246,604, filed Sep. 21, 2021, which is hereby incorporated by reference in its entirety.

### FIELD

The present invention relates to compounds and compositions for the production of hydrogel articles and, in particular, to compounds and compositions for additive manufacturing that provide biocompatible hydrogel articles having high biodegradability.

### BACKGROUND

Three-dimensional (3D) printers employ build materials, which are also known as inks, to form various 3D objects, articles, or parts in accordance with computer generated files. In some instances, the build material is solid at ambient temperatures and converts to liquid at elevated jetting temperatures. In other instances, the build material is liquid at ambient temperatures.

Build materials can comprise a variety of chemical species. Chemical species to include in a build material can be selected according to various considerations including, but not limited to, desired chemical and/or mechanical properties of the printed article and operating parameters of the 3D printing apparatus. Recently, for example, ink compositions for printing hydrogel articles have been developed. Hydrogels are unique materials, finding application in a wide variety of fields, including biomaterials. Hydrogel implants serving as scaffolds for tissue regeneration and/or various cellular therapies have attracted significant interest. Hydrogel scaffolds are often required to meet specific physical dimensions and exhibit certain microstructural features for cell/tissue interaction. Achieving these required tolerances with printable hydrogel compositions can be difficult, thereby limiting the efficacy of the hydrogel articles in medical implant applications.

### SUMMARY

The present disclosure contemplates curable compounds that can be used or otherwise incorporated in hydrogels or inks (or build materials or polymerizable liquids) for additive manufacturing applications as well as inks (or build materials or polymerizable liquids) which include the curable compounds. Methods of 3D printing, methods of making inks (or build materials or polymerizable liquids), and articles made from hydrogels or inks (or build materials or polymerizable liquids) are also described herein.

In one aspect, curable compounds are described herein. In some embodiments, such a compound has the structure of Formula (I) or the structure of Formula (II):

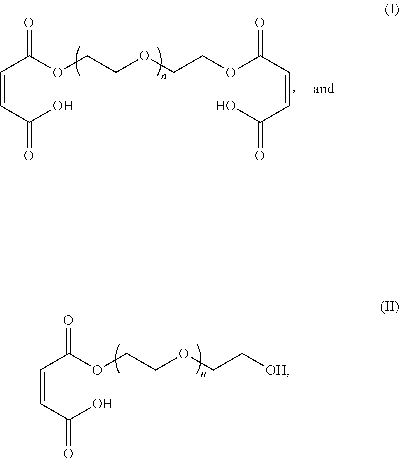

wherein n is an integer between 4 and 40 or between 4 and 20. Further, in some cases, a compound having the structure of Formula (I) or Formula (II) is a liquid at 25 degrees Celsius (° C.) and 1 atmosphere (atm).

In another aspect, hydrogels are described herein. Such a hydrogel can comprise a compound described hereinabove. Moreover, in some instances, a hydrogel described herein further comprises an acrylate component in addition to the compound of Formula (I) or Formula (II). A hydrogel described herein can also include additional components, as described further hereinbelow.

In still another aspect, inks or build materials or polymerizable liquids are described herein. It is to be understood that the terms “ink,” “build material,” and “polymerizable liquid” may be used interchangeably in the present disclosure. Further, such materials can be for hydrogel formation and/or use in a 3D printing system or method. In some embodiments, a build material for use in 3D printing described herein comprises one or more compounds having the structure(s) of Formula (I) and/or Formula (II) above.

In yet another aspect, methods of printing or forming a 3D article are described herein. In some embodiments, such a method comprises providing an ink, build material, or polymerizable liquid described herein, and printing and curing the ink, build material, or polymerizable liquid with light to form a hydrogel article, such as a medical implant. Additionally, in some cases, the ink, build material, or polymerizable liquid is provided in a layer-by-layer process.

In another aspect, printed 3D articles or objects are described herein, including such articles formed from an ink, build material, or polymerizable liquid described herein.

These and other embodiments are described in more detail in the detailed description which follows.

### DETAILED DESCRIPTION

Embodiments described herein can be understood more readily by reference to the following detailed description and examples. Compositions, apparatus and methods described herein, however, are not limited to the specific embodiments presented in the detailed description and examples. It should be recognized that these embodiments are merely illustrative of the principles of the present invention. Numerous modifications and adaptations will be readily apparent to those of skill in the art without departing from the spirit and scope of the invention.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 2 to 9, or 4.7 to 10.0, or 3.6 to 7.9, or 8 to 9.5.

All ranges disclosed herein are also to be considered to include the end points of the range, unless expressly stated otherwise. For example, a range of “between 5 and 10” should generally be considered to include the end points 5 and 10.

Further, when the phrase “up to” is used in connection with an amount or quantity, it is to be understood that the amount is at least a detectable amount or quantity. For example, a material present in an amount “up to” a specified amount can be present from a detectable amount and up to and including the specified amount.

Additionally, in any disclosed embodiment, the terms “substantially,” “approximately,” and “about” may be substituted with “within [a percentage] of” what is specified, where the percentage could be 0.1, 1, 5, or 10 percent.

It is also to be understood that the article “a” or “an” refers to “at least one,” unless the context of a particular use requires otherwise.

The terms “three-dimensional printing system,” “three-dimensional printer,” “printing,” and the like generally describe various solid freeform fabrication techniques for making three-dimensional articles or objects by selective deposition, jetting, fused deposition modeling, multijet modeling, and other additive manufacturing techniques now known in the art or that may be known in the future that use a build material or ink to fabricate three-dimensional articles or objects.

I. Curable Compounds

In one aspect, curable compounds are described herein. More particularly, the present disclosure contemplates curable compounds that can be used to form or otherwise be incorporated in hydrogels or inks (or build materials or polymerizable liquids) for 3D printing applications. One example embodiment of the present disclosure is a compound having the structure of Formula (I):

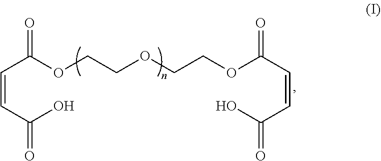

wherein n is an integer between 4 and 40.

Another example embodiment of the present disclosure is a compound having the structure of Formula (II):

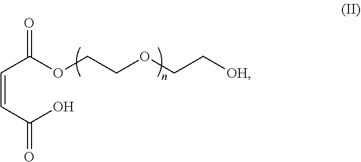

wherein n is an integer between 4 and 40.

Moreover, in some implementations, a compound has the structure of Formula (I) or Formula (II), wherein n is an integer between 4 and 14, between 4 and 20, between 6 and 30, between 10 and 40, or between 10 and 20. Other values of n are also possible.

In addition, a compound described herein, in some cases, is liquid at room temperature or at standard temperature and pressure conditions. For example, in some instances, a compound described herein is liquid at 25° C. and 1 atm or has a melting point less than or equal to about 25° C. In some embodiments, a compound described herein is liquid or has a melting point at 1 atm of 0-25° C., 0-22° C., 0-20° C., 5-25° C., 5-22° C., 5-20° C., 10-25° C., 10-22° C., 10-20° C., 15-25° C., 15-22° C., or 15-20° C. A compound having a melting point described herein, in some cases, can be well suited for hydrogel and/or build material formation.

A compound described herein can be made in any manner not inconsistent with the technical objectives of the present disclosure. For example, in some cases, a compound described herein is formed from the reaction of a poly(ethylene glycol) (PEG) and maleic anhydride (MA). An example reaction protocol is further described in the specific Examples below.

II. Hydrogels

In another aspect, hydrogels are described herein. As understood by one of ordinary skill in the art, a “hydrogel” can be considered to be a gel in which the liquid component is water or water-based. Additionally, a hydrogel described herein, in some embodiments, comprises or is defined by a network of one or more polymer(s) swollen with or encapsulating water or an aqueous solution or mixture. In some cases, a hydrogel described herein includes a network of polymers produced by curing or polymerizing a curable compound described herein and/or produced by curing or polymerizing one or more other polymerizable or curable species described herein, including in combination with a curable compound of Formula (I) or Formula (II) if desired.

In some embodiments, a hydrogel described herein comprises or is formed from a curable compound described hereinabove in Section I. Any such curable compound may be included in a hydrogel described herein. For example, in some embodiments, a hydrogel described herein comprises a compound having the structure of Formula (I). In other cases, a hydrogel described herein comprises a compound having the structure of Formula (II). In still other instances, a hydrogel described herein comprises a compound having the structure of Formula (I) and also a compound having the structure of Formula (II). In such cases, compounds having the structures of Formula (I) and Formula (II) can be present in a hydrogel described herein in any ratio not inconsistent with the technical objectives of the present disclosure. For example, in some embodiments, a compound having the structure of Formula (I) is present in an amount at least 2 times, at least 3 times, at least 5 times, or at least 10 times greater than the amount of a compound having the structure of Formula (II). In some instances, the ratio of Formula (I) to Formula (II) in a hydrogel described herein is at least 2:1, at least 3:1, at least 5:1, at least 10:1, or at least 100:1.

A curable compound described herein can be present in a hydrogel in any amount not inconsistent with the technical objectives of the present disclosure. For example, in some cases, a curable compound having the structure of Formula (I) or Formula (II) (or a cured or polymerized version thereof) is present in the hydrogel in an amount of 5 to 40 wt. %, based on the total weight of the hydrogel. In some implementations, the compound can be present in an amount or concentration of 5-35 wt. %, 8-33 wt. %, 10-35 wt. %, 10-30 wt. %, 10-20 wt. %, 10-15 wt. %, 12-25 wt. %, 15-35 wt. %, 20-30 wt. %, or 5-15 wt. %, based on the total weight of the hydrogel.

Moreover, in some implementations, a hydrogel described herein further comprises or is further formed from an acrylate component. Such an acrylate component can differ from the curable compound having the structure of Formula (I) or Formula (II). Any acrylate component not inconsistent with the technical objectives of the present disclosure may be used in such a hydrogel. It is particularly to be observed that an “acrylate” component, for reference purposes herein, can comprise one or more chemical species comprising at least one acrylate, methacrylate, acrylamide, or methacrylamide moiety or functional group. Additionally, it is to be understood that the term “(meth)acrylate” includes acrylate or methacrylate or a mixture or combination thereof. Similarly, the term “(meth)acrylamide” includes acrylamide or methacrylamide or a mixture or combination thereof.

In some cases, the acrylate component comprises a mono-, di-, tri-, or higher functional acrylate. In some instances, the acrylate component comprises an acrylate component described hereinbelow in Section III. For example, in some embodiments, the acrylate component comprises one or more hydroxyalkylacrylates, one or more polyethylene glycol acrylates or diacrylates, and/or one or more hydroxyalkylacrylamides. Other acrylate components may also be used.

An acrylate component described herein (or cured or polymerized version thereof) can be present in a hydrogel in any amount not inconsistent with the technical objectives of the present disclosure. For instance, in some cases, an acrylate component is present in the hydrogel in an amount of 15 to 50 wt. %, based on the total weight of the hydrogel. In some implementations, the acrylate component can be present in an amount or concentration of 15-40 wt. %, 15-30 wt. %, 20-50 wt. %, 20-45 wt. %, 25-40 wt. %, 25-35 wt. %, 30-45 wt. %, 30-50 wt. %, 30-40 wt. %, 35-50 wt. %, 35-45 wt. %, or 40-50 wt. %, based on the total weight of the hydrogel.

A hydrogel described herein can also comprise water in any amount not inconsistent with the technical objectives of the present disclosure. For example, in some cases, water is present in the hydrogel in an amount of 10 to 85 wt. % or 20 to 85 wt. %, based on the total weight of the hydrogel. In some implementations, water is present in an amount or concentration of 10-60 wt. %, 20-80 wt. %, 20-50 wt. %, 30-80 wt. %, 30-60 wt. %, 40-80 wt. %, 40-60 wt. %, 50-80 wt. %, or 50-70 wt. %, based on the total weight of the hydrogel.

It is further to be understood that the water (or the overall hydrogel) can have a pH of about 1 to about 7, about 3 to about 7, or about 4 to about 6. As understood by one of ordinary skill in the art, such a pH can be obtained, for example, by in the inclusion of a Bronsted-Lowry acid or base. For instance, in some cases, a strong acid or a strong base such as hydrochloric acid or sodium hydroxide, respectively, may be included in water (or the overall hydrogel) in a desired concentration to provide the desired pH, as understood by a person of ordinary skill. Other proton or hydroxide sources may also be used.

A hydrogel described herein can be made in any manner not inconsistent with the technical objectives of the present disclosure. For example, in some cases, a hydrogel described herein is formed by mixing the identified components of the hydrogel and curing or polymerizing the curable or polymerizable components (such as the curable compound(s) and the acrylate component). Such curing and polymerizing can be carried out, in some cases, in a manner described further hereinbelow, including in Section IV.

III. Polymerizable Liquids, Inks, or Build Materials

In another aspect, inks or build materials or polymerizable liquids are described herein. Such materials can be for hydrogel formation and/or use in a 3D printing system or method. In some embodiments, a polymerizable liquid or build material for use in 3D printing described herein comprises a compound having the structure of Formula (I) or Formula (II), wherein n is an integer between 4 and 40. Such a build material may further comprise an acrylate component, and water.

In certain implementations, the polymerizable liquid comprises a compound having the structure of Formula (I) and also a compound having the structure of Formula (II). In other instances, the polymerizable liquid comprises a compound having the structure of Formula (I) but not a compound having the structure of Formula (II). In some cases, the polymerizable liquid may include one or more compounds having the structure of Formula (I) while also including substantially no amount of compound having the structure of Formula (II), or while including less than 5 wt. %, less than 3 wt. %, or less than 1 wt. % compound having the structure of Formula (II), based on the total weight of the polymerizable liquid. In some instances, the ratio of compounds having the structure of Formula (I) to compounds having the structure of Formula (II) (by weight) is at least 20:1, at least 10:1, at least 5:1, or at least 3:1. In some embodiments, the ratio of Formula (I) compounds to Formula (II) compounds is between 100:1 and 100:0, between 100:1 and 50:1, between 100:1 and 10:1, between 50:1 and 20:1, between 50:1 and 10:1, between 20:1 and 5:1, or between 10:1 and 2:1.

Further, it should be understood that polymerizable liquids described herein may include only one or multiple compounds having the structure of Formula (I). For example, in some implementations, the polymerizable liquid can include multiple compounds having the structure of Formula (I) such as a first compound having the structure of Formula (I) where n=4 and a second compound having the structure of Formula (I) where n=6. Alternatively, in other implementations, the polymerizable liquid can only include a single compound having the structure of Formula (I), such as a compound having the structure of Formula (I) where n=10, where substantially no amount or no detectable amount of other compounds having the structure of Formula (I) (e.g., having n not equal to 10) are present in the polymerizable liquid. The same can be the case for compounds having the structure of Formula (II), in an analogous manner as described in this paragraph for Formula (I).

In general, the compound having the structure of Formula (I) or Formula (II) can be present in a polymerizable liquid described herein in any amount not inconsistent with the objectives of the present disclosure. In some embodiments, for instance, the compound having the structure of Formula (I) or Formula (II) is present in an amount of 4-40 wt. %, based on total weight of the polymerizable liquid. In some cases, the compound having the structure of Formula (I) or Formula (II) is present in the polymerizable liquid in an amount of 5-35 wt. %, 8-33 wt. %, 10-35 wt. %, 10-30 wt. %, 10-20 wt. %, 10-15 wt. %, 12-25 wt. %, 15-35 wt. %, 20-30 wt. %, or 5-15 wt. %, based on the total weight of the polymerizable liquid.

Turning now to other components of the polymerizable liquid, a polymerizable liquid described herein, in some embodiments, further comprises an acrylate component. Any acrylate component not inconsistent with the technical objectives of the present disclosure may be used. It is particularly to be observed that an “acrylate” component, for reference purposes herein, can comprise one or more chemical species comprising at least one acrylate, methacrylate, acrylamide, or methacrylamide moiety or functional group. Additionally, it is to be understood that the term “(meth)acrylate” includes acrylate or methacrylate or a mixture or combination thereof, and the term “(meth)acrylamide” includes acrylamide or methacrylamide or a mixture or combination thereof.

In some embodiments, the acrylate component comprises hydrophilic mono-, di-, and/or tri(meth)acrylate species. The acrylate component, for example, can comprise one or more of hydroxyalkyl(meth)acrylates (e.g., hydroxyethylacrylate), hydroxyalkyl(meth)acrylamides (e.g., N-hydroxyethylacrylamide), ethoxylated trimethylol propane triacrylate, acryloyl morpholine, and various combinations or mixtures thereof. In some embodiments, hydroxyalkyl(meth)acrylates include hydroxyethyl(meth)acrylate, hydroxypropyl(meth)acrylate, hydroxybutyl(meth)acrylate, and/or mixtures thereof.

The acrylate component of a polymerizable liquid described herein may also include a polyethylene glycol diacrylate (PEGDA). With reference to the polyethylene glycol diacrylate component as used herein, the polyethylene glycol diacrylates can comprise a single polyethylene glycol diacrylate species or polyethylene glycol diacrylate species of differing molecular weights. In some embodiments, species of the polyethylene glycol diacrylate component have a weight average molecular weight of 0.1 kDa to 20 kDa. Molecular weight of individual species of polyethylene glycol diacrylate, for example, can fall within one or more ranges set forth in Table 1.

TABLE 1
Polyethylene Glycol Diacrylate Molecular Weight (kDa)
0.1-1
0.5-1
3-10
10-20
0.5-5

Any combination or mixture of polyethylene glycol diacrylates of differing molecular weight is contemplated. In some embodiments, the polyethylene glycol diacrylate component comprises a mixture of two of more polyethylene diacrylate species each having molecular weight from 0.5 to 5 kDa. Specific composition of the polyethylene glycol diacrylate component can be selected according to several considerations including, but not limited to, crosslink density, elasticity, tensile strength, and/or mesh size of the resulting hydrogel article.

The polyethylene glycol diacrylate component can be present in the polymerizable liquid in any amount not inconsistent with the technical objectives described herein. In some embodiments, the polyethylene glycol diacrylate component is present in an amount of 5-60 wt. %, based on total weight of the polymerizable liquid. For example, the polyethylene glycol diacrylate component can comprise 5-30 wt. % polyethylene glycol diacrylate species having molecular weight of 3-5 kDa and 2-20 wt. % polyethylene glycol diacrylate species having molecular weight of 0.1-1 kDa.

It is to be understood that the acrylate component of a polymerizable liquid described herein can include a combination of acrylate species. For example, in some cases, the acrylate component is selected from one or more hydroxyalkylacrylates, one or more polyethylene glycol acrylates, one or more polyethylene glycol diacrylates, one or more hydroxyalkylacrylamides, or a combination thereof. In certain polymerizable liquids, the acrylate component can include only one hydroxyalkylacrylate. In other polymerizable liquids, the acrylate component can include a plurality (more than one) of hydroxyalkylacrylates. In still other polymerizable liquids, the acrylate component can include one polyethylene glycol acrylate and one hydroxyalkylacrylamide. Thus, the present disclosure contemplates many combinations and compositions of the acrylate component that can be included in example implementations, though they are not explicitly enumerated herein.

In general, the acrylate component of a polymerizable liquid described herein can be present in the polymerizable liquid in any amount not inconsistent with the technical objectives of the present disclosure. In some embodiments, for example, the acrylate component is present in an amount or concentration of no less than 15 wt. % and no greater than 50 wt. %, based on total weight of the polymerizable liquid. In some implementations, the acrylate component is present in an amount or concentration of 20-45 wt. %, 25-40 wt. %, 30-40 wt. %, 30-50 wt. %, 35-50 wt. %, or 40-50 wt. %, based on total weight of the polymerizable liquid.

A polymerizable liquid described herein can also comprise water. Water can be present in any amount not inconsistent with the technical objectives of the present disclosure. For example, in some cases, water is present in the polymerizable liquid in an amount of 10 to 85 wt. % or 20 to 85 wt. %, based on the total weight of the polymerizable liquid. In some implementations, water is present in an amount or concentration of 10-60 wt. %, 20-80 wt. %, 20-50 wt. %, 30-80 wt. %, 30-60 wt. %, 40-80 wt. %, 40-60 wt. %, 50-80 wt. %, or 50-70 wt. %, based on the total weight of the polymerizable liquid.

It is further to be understood that the water (or the overall polymerizable liquid) can have a pH of about 1 to about 7, about 3 to about 7, or about 4 to about 6. As understood by one of ordinary skill in the art, such a pH can be obtained, for example, by in the inclusion of a Bronsted-Lowry acid or base. For instance, in some cases, a strong acid or a strong base such as hydrochloric acid or sodium hydroxide, respectively, may be included in water (or the overall polymerizable liquid) in a desired concentration to provide the desired pH, as understood by a person of ordinary skill. Other proton or hydroxide sources may also be used.

In some implementations, the polymerizable liquid can further include one or more additional polymerizable or curable materials differing from the compound having the structure of Formula (I) or Formula (II) and differing from the acrylate component. Such an additional polymerizable or curable material, in some embodiments, can comprise any chemical species having a curable or polymerizable moiety, such as an ethyleneically unsaturated moiety that could participate in a curing or polymerization reaction with the curable compound and/or acrylate component of the polymerizable liquid. Further, in some instances, such an additional polymerizable or curable material is hydrophilic. In some embodiments, the one or more additional polymerizable or curable materials can be present in an amount of 1-20 wt. %, based on total weight of the polymerizable liquid.

In some embodiment, a polymerizable liquid described herein further comprises a colorant or an emitter compound or compounds. The emitter compound can include a structure such that on exposure to a certain wavelength(s) of radiation, the emitter compound will produce a visible signal. In some implementations, the emitter compound(s) can include one or more colorants that produce a visible wavelength (e.g., red, green, blue, etc.) on exposure to natural light. When incorporated in the polymerizable liquid, the colorant(s) can be present in an amount of 0.1-5 wt. %, 0.1-3 wt. %, 0.1-2 wt. %, 0.1-1 wt. %, 0.1-0.5 wt. %, 0.5-5 wt. %, 0.5-4 wt. %, or 0.5-3 wt. %, based on total weight of the polymerizable liquid.

A polymerizable liquid described herein, in some embodiments, also comprises a photoinitiator component for initiating polymerization of one or more components of the liquid upon exposure to light of the proper wavelength. In some embodiments, the photoinitiator component can initiate polymerization/crosslinking of the curable compound. As described above, the acrylate component (and roptionally one or more additional polymerizable or curable materials) may also participate in this polymerization.

Any photoinitiator not inconsistent with the objectives of the present disclosure can be used. In some embodiments, a photoinitiator comprises an alpha-cleavage type (unimolecular decomposition process) photoinitiator or a hydrogen abstraction photosensitizer-tertiary amine synergist, operable to absorb light preferably between about 250 nm and about 420 nm or between about 300 nm and about 385 nm, to yield free radical(s).

Examples of alpha cleavage photoinitiators are Irgacure 184 (CAS 947-19-3), Irgacure 369 (CAS 119313-12-1), and Irgacure 819 (CAS 162881-26-7). An example of a photosensitizer-amine combination is Darocur BP (CAS 119-61-9) with diethylaminoethylmethacrylate.

In addition, in some instances, suitable photoinitiators comprise benzoins, including benzoin, benzoin ethers, such as benzoin methyl ether, benzoin ethyl ether and benzoin isopropyl ether, benzoin phenyl ether and benzoin acetate, acetophenones, including acetophenone, 2,2-dimethoxyacetophenone and 1,1-dichloroacetophenone, benzil, benzil ketals, such as benzil dimethyl ketal and benzil diethyl ketal, anthraquinones, including 2-methylanthraquinone, 2-ethylanthraquinone, 2-tert-butylanthraquinone, 1-chloroanthraquinone and 2-amylanthraquinone, triphenylphosphine, benzoylphosphine oxides, for example 2,4,6-trimethylbenzoyldiphenylphosphine oxide (Lucirin TPO), benzophenones, such as benzophenone and 4,4′-bis(N,N′-dimethylamino)benzophenone, thioxanthones and xanthones, acridine derivatives, phenazine derivatives, quinoxaline derivatives or 1-phenyl-1,2-propanedione, 2-O-benzoyl oxime, 1-aminophenyl ketones or 1-hydroxyphenyl ketones, such as 1-hydroxycyclohexyl phenyl ketone, phenyl 1-hydroxyisopropyl ketone and 4-isopropylphenyl 1-hydroxyisopropyl ketone.

Suitable photoinitiators can also comprise those operable for use with a HeCd laser radiation source, including acetophenones, 2,2-dialkoxybenzophenones and 1-hydroxyphenyl ketones, such as 1-hydroxycyclohexyl phenyl ketone or 2-hydroxyisopropyl phenyl ketone (=2-hydroxy-2,2-dimethylacetophenone). Additionally, in some cases, suitable photoinitiators comprise those operable for use with an Ar laser radiation source including benzil ketals, such as benzil dimethyl ketal. In some embodiments, a photoinitiator comprises an α-hydroxyphenyl ketone, benzil dimethyl ketal or 2,4,6-trimethylbenzoyldiphenylphosphine oxide or a mixture thereof.

Another class of suitable photoinitiators, in some instances, comprises ionic dye-counter ion compounds capable of absorbing actinic radiation and generating free radicals for polymerization initiation. In some embodiments, polymerizable liquids containing ionic dye-counter ion compounds can be polymerized upon exposure to visible light within the adjustable wavelength range of about 400 nm to about 700 nm. Ionic dye-counter ion compounds and their mode of operation are disclosed in EP-A-0 223 587 and U.S. Pat. Nos. 4,751,102; 4,772,530; and 4,772,541.

A photoinitiator can be present in a polymerizable liquid described herein in any amount not inconsistent with the objectives of the present disclosure. In some embodiments, a photoinitiator is present in an amount of up to about 5 wt. %, based on the total weight of the polymerizable liquid. In some cases, a photoinitiator is present in an amount ranging from about 0.1 wt. % to about 5 wt. %, an amount of about 0.1 wt. % to about 3 wt. %, an amount of about 0.5 wt. % to about 2.5 wt. %, or an amount of about 1 wt. % to about 3 wt. %.

Moreover, in some implementations, the polymerizable liquid can further include one or more sensitizers. A sensitizer can be added to increase the effectiveness of one or more photoinitiators that may also be present. Any sensitizer not inconsistent with the objectives of the present disclosure may be used. In some cases, a sensitizer comprises isopropylthioxanthone (ITX) or 2-chlorothioxanthone (CTX).

A sensitizer can be present in the polymerizable liquid in any amount not inconsistent with the objectives of the present disclosure. In some embodiments, a sensitizer is present in an amount ranging from about 0.1 wt. % to about 3 wt. %, from about 0.1 wt. % to about 2 wt. %, from about 0.5 wt. % to about 2 wt. %, or from about 0.5 wt. % to about 1 wt. %, based on the total weight of the polymerizable liquid.

In some implementations, one or more UV-absorbers and/or light stabilizers can be present in the polymerizable liquid at an effective concentration. For example, one or more UV-absorbers and/or light stabilizers can be present in an amount of 0.1-2 wt. %, based on the total weight of the polymerizable liquid. In some embodiments, UV-absorbers and/or light stabilizers are commercially available from BASF of Florham Park, New Jersey under the TINUVIN® trade-designation, and from QCR Solutions Corporation under the UV386 trade designation.

Polymerizable liquids or inks described herein can have a variety of properties in a cured or uncured state, including properties related to the microstructure of the ink, which may be a complex mixture or other complex material system. In some embodiments, such structural features or other properties relate to the polymerizable liquid in a cured or polymerized state. An ink (or build material or polymerizable liquid) in a “cured” or “polymerized” state, as used throughout the present disclosure, comprises an ink (or build material or polymerizable liquid) that includes a curable material or polymerizable component that has been at least partially cured, i.e., at least partially polymerized and/or cross-linked. For instance, in some cases, a cured ink (or build material or polymerizable liquid) is at least about 70% polymerized or cross-linked or at least about 80% polymerized or cross-linked. In some embodiments, a cured ink (or build material or polymerizable liquid) is at least about 85%, at least about 90%, at least about 95%, at least about 98%, or at least 99% polymerized or cross-linked. In some instances, a cured ink (or build material or polymerizable liquid) is between about 80% and about 99% polymerized or cross-linked. The degree of polymerization or cross-linking can be determined using any protocol or method not inconsistent with the technical objectives of the present disclosure, such as by determining the percentage of monomers incorporated into the polymer network (e.g., based on molecular weight of the polymer compared to the molecular weight of the monomer, or based on the total polymer mass compared to the theoretical maximum of the total polymer mass) or by determining the amount of unincorporated monomers. When more than one method is used to determine a degree of polymerization or cross-linking, the results of the methods can be averaged to obtain a percentage described herein. It is further to be understood that the degree of polymerization or cross-linking described herein is different than “degree of polymerization” defined as the number of repeating units in a polymer molecule.

In some embodiments, an ink (or build material or polymerizable liquid) described herein when cured or polymerized has an elongation at break of greater than 150%, when measured according to the method of Example 6. For example, certain articles formed from polymerization of a polymerizable liquid in accordance with the present disclosure can have an elongation at break of 150-300%, 150-275%, 150-250%, 200-275%, or 200-250%, when measured according to the method of Example 6.

Another example property of certain polymerizable liquids (or inks or build materials) upon polymerization can include a percent swelling in phosphate buffered saline of less than 30%, less than 20%, less than 15%, or less than 10%, when measured according to the method of Example 7. For example, some articles formed from polymerization of a polymerizable liquid in accordance with the present disclosure can have a percent swelling in the range of 0-30%, 0-25%, 0-20%, 0-15%, 1-30%, 1-20%, 1-15%, 5-30%, 5-25%, 5-20%, 5-15%, 10-30%, 10-20%, 10-15%, 15-30%, 15-25%, or 15-20%, when measured according to the method of Example 7.

Inks (or build materials or polymerizable liquids) described herein can be produced in any manner not inconsistent with the objectives of the present disclosure. In some embodiments, for instance, a method for the preparation of an ink (or build material or polymerizable liquid) described herein comprises the steps of mixing the components of the ink, optionally melting the mixture, and filtering the (optionally molten) mixture. In some cases, the components are mixed and optionally melted at a temperature between about 25° C. and about 35° C., or at a temperature in the range of 25-55° C., 35-65° C., or 45-75° C. In some instances in which it is desirable or necessary to melt one or more solid components of the ink, mixing and/or melting can be carried about a temperature in a range from about 75° C. to about 85° C. In some embodiments, an ink described herein is produced by placing all components of the ink in a reaction vessel, optionally heating the resulting mixture, and stirring the resulting mixture at a temperature between about 25° C. and about 75° C. or a temperature ranging from about 75° C. to about 85° C. The stirring (and optionally the heating) are continued until the mixture attains a substantially homogenized liquid (or molten) state. In general, the liquid (or molten) mixture can be filtered while in a flowable state to remove any large undesirable particles that may interfere with jetting or extrusion or other printing process. The filtered mixture can then be cooled to ambient temperatures (if cooling is needed) and stored until ready for use in a 3D printing system.

IV. Methods of Forming a 3D Article

In another aspect, methods of forming or “printing” a 3D article or object (such as a hydrogel article or object) by additive manufacturing are described herein. Methods of forming a 3D article or object described herein can include forming the 3D article from a plurality of layers of an ink (or build material or polymerizable liquid) described herein in a layer-by-layer manner (such as in NAJP (multijet printing) or SLA (stereolithography) printing methods). For example, in some instances, an NAJP method of printing a 3D article comprises selectively depositing layers of an ink described herein in a fluid state onto a substrate, such as a build pad of a 3D printing system. The method can further comprise further curing (e.g., photocuring) the ink. Moreover, curing can comprise polymerizing one or more polymerizable moieties or functional groups of one or more components of the ink. In some cases, a layer of deposited ink is cured prior to the deposition of another or adjacent layer of ink. Additionally, curing one or more layers of deposited ink, in some embodiments, is carried out by exposing the one or more layers to electromagnetic radiation, such as UV light, visible light, or infrared light, as described above. In addition, in some embodiments, such a method further comprises supporting at least one of the layers of the ink with a support material, before or after curing. Any support material not inconsistent with the objectives of the present disclosure may be used, as described further below.

Alternatively, a method of printing a 3D article comprises retaining an ink in a fluid state in a container; selectively applying energy to the ink in the container to solidify at least a portion of a first fluid layer of the ink, thereby forming a first solidified layer that defines a first cross-section of the article; raising or lowering the first solidified layer to provide a second fluid layer of the ink at a surface of the fluid ink in the container; and selectively applying energy to the ink in the container to solidify at least a portion of the second fluid layer of the ink, thereby forming a second solidified layer that defines a second cross-section of the article, the first cross-section and the second cross-section being bonded to one another in a z-direction. Moreover, in some such embodiments, selectively applying energy to the ink in the container comprises photocuring the ink.

Further, in some embodiments of methods described herein, one or more layers of an ink described herein has a thickness of about 10 μm to about 100 μm, about 10 μm to about 80 μm, about 10 μm to about 50 μm, about 20 μm to about 100 μm, about 20 μm to about 80 μm, or about 20 μm to about 40 μm. Other thicknesses are also possible.

Methods of forming a 3D article by additive manufacturing can also include forming the object in a manner other than a layer-by-layer manner.

Additionally, any ink (or build material or polymerizable liquid) described hereinabove in Section III or in the specific Examples below may be used in a method described herein. For example, in some cases, a method described herein can include providing a polymerizable liquid to a print bed, the polymerizable liquid including a curable compound, an acrylate component, and water. The method can further comprise curing at least a portion of the polymerizable liquid. More particularly, a composition described herein can be provided (e.g., by a 3D printing system) to a print area and at least a portion of the provided composition cured (e.g., using electromagnetic radiation directed at the portion of the provided composition).

Moreover, a process of curing/polymerizing described herein can be performed such that the polymerizable liquid (or ink or build material) forms an article having physical and/or material properties in accordance with example embodiments described herein. Further details regarding various methods, including “material deposition” methods (such as MjP) or “vat polymerization” methods (such as SLA), are provided below.

A. Material Deposition Methods

In a material deposition method, one or more layers of an ink described herein are selectively deposited onto a substrate and cured. Curing of the ink may occur after selective deposition of one layer, each layer, several layers, or all layers of the ink.

In some instances, an ink described herein is selectively deposited in a fluid state onto a substrate, such as a build pad of a 3D printing system. Selective deposition may include, for example, depositing the ink according to preselected CAD (computer-aided design) parameters. For example, in some embodiments, a CAD file drawing corresponding to a desired 3D article to be printed is generated and sliced into a sufficient number of horizontal slices. Then, the ink is selectively deposited, layer by layer, according to the horizontal slices of the CAD file drawing to print the desired 3D article. A “sufficient” number of horizontal slices is the number necessary for successful printing of the desired 3D article, e.g., to produce it accurately and precisely.

Further, in some embodiments, a preselected amount of ink described herein is heated to the appropriate temperature and jetted through a print head or a plurality of print heads of a suitable inkjet printer to form a layer on a print pad in a print chamber. In some cases, each layer of ink is deposited according to preselected CAD parameters. A suitable print head to deposit the ink, in some embodiments, is a piezoelectric print head. Additional suitable print heads for the deposition of ink and support material described herein are commercially available from a variety of ink jet printing apparatus manufacturers. For example, Xerox, Hewlett Packard, or Ricoh print heads may be used in some instances.

Additionally, in some embodiments, an ink described herein remains substantially fluid upon deposition. Alternatively, in other instances, the ink exhibits a phase change upon deposition and/or solidifies upon deposition. Moreover, in some cases, the temperature of the printing environment can be controlled so that the jetted droplets of ink solidify on contact with the receiving surface. In other embodiments, the jetted droplets of ink do not solidify on contact with the receiving surface, remaining in a substantially fluid state. Additionally, in some instances, after each layer is deposited, the deposited material is planarized and cured with electromagnetic (e.g., UV, visible, or infrared light) radiation prior to the deposition of the next layer. Optionally, several layers can be deposited before planarization and curing, or multiple layers can be deposited and cured followed by one or more layers being deposited and then planarized without curing. Planarization corrects the thickness of one or more layers prior to curing the material by evening the dispensed material to remove excess material and create a uniformly smooth exposed or flat up-facing surface on the support platform of the printer. In some embodiments, planarization is accomplished with a wiper device, such as a roller, which may be counter-rotating in one or more printing directions but not counter-rotating in one or more other printing directions. In some cases, the wiper device comprises a roller and a wiper that removes excess material from the roller. Further, in some instances, the wiper device is heated. It should be noted that the consistency of the jetted ink described herein prior to curing, in some embodiments, should desirably be sufficient to retain its shape and not be subject to excessive viscous drag from the planarizer.

Moreover, a support material, when used, can be deposited in a manner consistent with that described hereinabove for the ink. The support material, for example, can be deposited according to the preselected CAD parameters such that the support material is adjacent or continuous with one or more layers of the ink. Jetted droplets of the support material, in some embodiments, solidify or freeze on contact with the receiving surface. In some cases, the deposited support material is also subjected to planarization, curing, or planarization and curing. Any support material not inconsistent with the objectives of the present disclosure may be used.

Layered deposition of the ink and support material can be repeated until the 3D article has been formed. In some embodiments, a method of printing a 3D article further comprises removing the support material from the ink.

Curing of the ink may occur after selective deposition of one layer of ink, of each layer of ink, of several layers of ink, or of all layers of the ink necessary to print the desired 3D article. In some embodiments, a partial curing of the deposited ink is performed after selective deposition of one layer of ink, each layer of ink, several layers of ink, or all layers of the ink necessary to print the desired 3D article. A “partially cured” ink, for reference purposes herein, is one that can undergo further curing. For example, a partially cured ink is up to about 30% polymerized or cross-linked or up to about 50% polymerized or cross-linked. In some embodiments, a partially cured ink is up to about 60%, up to about 70%, up to about 80%, up to about 90%, or up to about 95% polymerized or cross-linked.

Partial curing of the deposited ink can include irradiating the ink with an electromagnetic radiation source or photocuring the ink (including with curing radiation described hereinabove). Any electromagnetic radiation source not inconsistent with the objectives of the present disclosure may be used, e.g., an electromagnetic radiation source that emits UV, visible or infrared light. For example, in some embodiments, the electromagnetic radiation source can be one that emits light having a wavelength from about 300 nm to about 900 nm, e.g., a Xe arc lamp.

Further, in some embodiments, a post-curing is performed after partially curing is performed. For example, in some cases, post-curing is carried out after selectively depositing all layers of the ink necessary to form a desired 3D article, after partially curing all layers of the ink, or after both of the foregoing steps have been performed. Moreover, in some embodiments, post-curing comprises photocuring. Again, any electromagnetic radiation source not inconsistent with the objectives of the present disclosure may be used for a post-curing step described herein. For example, in some embodiments, the electromagnetic radiation source can be a light source that has a higher energy, a lower energy, or the same energy as the electromagnetic radiation source used for partial curing. In some cases wherein the electromagnetic radiation source used for post-curing has a higher energy (i.e., a shorter wavelength) than that used for partial curing, a Xe arc lamp can be used for partial curing and a Hg lamp can be used for post-curing.

Additionally, after post-curing, in some cases, the deposited layers of ink are at least about 80% polymerized or cross-linked or at least about 85% polymerized or cross-linked. In some embodiments, the deposited layers of ink are at least about 90%, at least about 95%, at least about 98%, or at least about 99% polymerized or cross-linked. In some instances, the deposited layers of ink are about 80-100%, about 80-99%, about 80-95%, about 85-100%, about 85-99%, about 85-95%, about 90-100%, or about 90-99% polymerized or cross-linked.

B. Vat Polymerization Methods

It is also possible to form a 3D article from an ink described herein using a vat polymerization method, such as an SLA method. Thus, in some cases, a method of printing a 3D article described herein comprises retaining an ink described herein in a fluid state in a container and selectively applying energy (particularly, for instance, curing radiation) to the ink in the container to solidify at least a portion of a fluid layer of the ink, thereby forming a solidified layer that defines a cross-section of the 3D article. Additionally, a method described herein can further comprise raising or lowering the solidified layer of ink to provide a new or second fluid layer of unsolidified ink at the surface of the fluid ink in the container, followed by again selectively applying energy (e.g., the curing radiation) to the ink in the container to solidify at least a portion of the new or second fluid layer of the ink to form a second solidified layer that defines a second cross-section of the 3D article. Further, the first and second cross-sections of the 3D article can be bonded or adhered to one another in the z-direction (or build direction corresponding to the direction of raising or lowering recited above) by the application of the energy for solidifying the ink. Moreover, in some instances, the electromagnetic radiation has an average wavelength of 300-900 nm, and in other embodiments the electromagnetic radiation has an average wavelength that is less than 300 nm. In some cases, the curing radiation is provided by a computer controlled laser beam. In addition, in some cases, raising or lowering a solidified layer of ink is carried out using an elevator platform disposed in the container of fluid ink. A method described herein can also comprise planarizing a new layer of fluid ink provided by raising or lowering an elevator platform. Such planarization can be carried out, in some cases, by a wiper or roller.

It is further to be understood that the foregoing process can be repeated a desired number of times to provide the 3D article. For example, in some cases, this process can be repeated “n” number of times, wherein n can be up to about 100,000, up to about 50,000, up to about 10,000, up to about 5000, up to about 1000, or up to about 500. Thus, in some embodiments, a method of printing a 3D article described herein can comprise selectively applying energy (e.g., curing radiation) to an ink in a container to solidify at least a portion of an nth fluid layer of the ink, thereby forming an nth solidified layer that defines an nth cross-section of the 3D article, raising or lowering the nth solidified layer of ink to provide an (n+1)th layer of unsolidified ink at the surface of the fluid ink in the container, selectively applying energy to the (n+1)th layer of ink in the container to solidify at least a portion of the (n+1)th layer of the ink to form an (n+1)th solidified layer that defines an (n+1)th cross-section of the 3D article, raising or lowering the (n+1)th solidified layer of ink to provide an (n+2)th layer of unsolidified ink at the surface of the fluid ink in the container, and continuing to repeat the foregoing steps to form the 3D article. Further, it is to be understood that one or more steps of a method described herein, such as a step of selectively applying energy (e.g., curing radiation) to a layer of ink, can be carried out according to an image of the 3D article in a computer-readable format. General methods of 3D printing using stereolithography are further described, inter alia, in U.S. Pat. Nos. 5,904,889 and 6,558,606.

Performing a printing process described above can provide a printed 3D article from an ink described herein that has a high feature resolution. The “feature resolution” of an article, for reference purposes herein, can be the smallest controllable physical feature size of the article. The feature resolution of an article can be described in terms of a unit of distance such as microns (μm), or in terms of dots per inch (dpi). As understood by one of ordinary skill in the art, a higher feature resolution corresponds to a higher dpi value but a lower distance value in μm. In some cases, an article formed by depositing or solidifying an ink described herein can have a feature resolution of about 500 μm or less, about 200 μm or less, about 100 μm or less, or about 50 μm or less, including at elevated temperatures. In some embodiments, an article has a feature resolution between about 50 μm and about 500 μm, between about 50 μm and about 200 μm, between about 50 μm and about 100 μm, or between about 100 μm and about 200 μm. Correspondingly, in some instances, an article described herein has a feature resolution of at least about 100 dpi, at least about 200 dpi, at least about 250 dpi, at least about 400 dpi, or at least about 500 dpi. In some cases, the feature resolution of an article is between about 100 dpi and about 600 dpi, between about 100 dpi and about 250 dpi, or between about 200 dpi and about 600 dpi.

In a vat polymerization method such as described above, the ink may be partially cured as described in Section IV.A above. For example, in some embodiments, selectively applying energy to the ink in the container to solidify at least a portion of a fluid layer of the ink may include partially curing at least a portion of a fluid layer of the ink. In other embodiments, partial curing of at least a portion of a fluid layer of the ink may occur after a first layer of the ink is provided and solidified, before or after a second layer of the ink is provided or solidified, or before or after one, several, or all subsequent layers of the ink are provided or solidified.

Additionally, in some embodiments of a vat polymerization method described herein, after partial curing or after the desired 3D article is formed, post-curing as described in Section IV.A above may be performed. The desired 3D article may be, for example, an article that corresponds to the design in a CAD file.

V. Printed Articles

In another aspect, printed 3D articles are described herein. In some embodiments, a printed 3D article is formed from an ink (or build material or polymerizable liquid) described herein. Any ink (or build material or polymerizable liquid) described hereinabove in Section III may be used. For example, in some cases, the ink comprises a curable compound having the structure of Formula (I) or Formula (II), an acrylate component, and water. Further, in some cases, a printed 3D article described herein is formed primarily from a polymer network resulting from the curing or polymerization of the curable compound and acrylate component. An article described herein can also be a hydrogel article or an article formed from a hydrogel and exhibiting hydrogel properties.

Hydrogel articles printed according to methods described herein can find application in a variety of fields, including the medical field. The hydrogel articles, for example, can be medical implants. The hydrogel medical implants can be employed for tissue regeneration and/or serve as scaffolds for cellular seeding and/or growth. It is also possible to form mimics of biological tissue or organs using a hydrogel or polymerizable liquid described herein.

### EXAMPLES

Some specific embodiments of curable compounds, hydrogels, inks (or build materials or polymerizable liquids), methods, and articles are further illustrated in the following non-limiting Examples.

### Example 1

A curable compound denoted as MA-PEG200-MA was prepared as follows. It is to be understood that this species has the structure of Formula (I), in which n corresponds to a PEG moiety having a weight average molecular weight of approximately 200.

To a 2-neck 1-liter round bottom flask with a Teflon coated stir magnet, stopper, and 24/40 adapter was added 216.66 grams PEG 200. The round bottom flask (reaction vessel) was placed in a 65° C. oil bath with condenser in place. Via glass funnel, transferred 218.92 grams maleic anhydride to reaction vessel. Stirred at 65° C. until thoroughly dissolved and homogenous. Ran drierite house nitrogen over the headspace of the round bottom flask for approximately 30 seconds prior to stopper placement with drierite house nitrogen gently running over the top of the condenser.

After 3 hours, collected initial infrared (IR) spectroscopy time point. Continued 65° C. reflux for approximately 36 hours. Discontinued reflux after product confirmation by IR.

### Example 2

A curable compound denoted as MA-PEG400-MA is prepared as follows. It is to be understood that this species has the structure of Formula (I), in which n corresponds to a PEG moiety having a weight average molecular weight of approximately 400.

To a 2-neck 1-liter round bottom flask with a Teflon coated stir magnet, stopper, and 24/40 adapter is added 430 grams PEG 400. The round bottom flask is placed in a 65° C. oil bath with condenser in place. Via glass funnel, 220 grams maleic anhydride is transferred to the reaction vessel. Stirring is carried out at 65° C. until the flask contents are thoroughly dissolved and homogenous. Nitrogen gas is provided over the headspace of the round bottom flask for approximately 30 seconds prior to stopper placement, with nitrogen gently running over the top of the condenser.

After 3 hours, IR spectroscopy is carried out on a sample aliquot from the reaction vessel to provide an initial IR time point. Reflux at 65° C. is continued for approximately 36 hours. Reflux is discontinued after product confirmation by IR.

### Example 3

A curable compound denoted as MA-PEG600-MA is prepared as follows. It is to be understood that this species has the structure of Formula (I), in which n corresponds to a PEG moiety having a weight average molecular weight of approximately 600.

To a 2-neck 2-liter round bottom flask with a Teflon coated stir magnet, stopper, and 24/40 adapter is added 650 grams PEG 600. The round bottom flask is placed in a 65° C. oil bath with condenser in place. Via glass funnel, 220 grams maleic anhydride is transferred to the reaction vessel. Stirring is carried out at 65° C. until the flask contents are thoroughly dissolved and homogenous. Inert gas (nitrogen, dried) is provided over the headspace of the round bottom flask for approximately 30 seconds prior to stopper placement, with dried inert gas (nitrogen) gently running over the top of the condenser.

After 3 hours, IR spectroscopy is carried out on a sample aliquot from the reaction vessel to provide an initial IR time point. Reflux at 65° C. is continued for approximately 36 hours. Reflux is discontinued after product confirmation by IR.

### Example 4

A curable compound denoted as MA-PEG1000-MA is prepared as follows. It is to be understood that this species has the structure of Formula (I), in which n corresponds to a PEG moiety having a weight average molecular weight of approximately 1000.

To a 2-neck 2-liter round bottom flask with a Teflon coated stir magnet, stopper, and 24/40 adapter is added 1,080 grams PEG 1000. The round bottom flask is placed in a 65° C. oil bath with condenser in place. Via glass funnel, 220 grams maleic anhydride is transferred to the reaction vessel. Stirring is carried out at 65° C. until the flask contents are thoroughly dissolved and homogenous. Dried nitrogen is provided over the headspace of the round bottom flask for approximately 30 seconds prior to stopper placement, with dried nitrogen gently running over the top of the condenser.

After 3 hours, IR spectroscopy is carried out on a sample aliquot from the reaction vessel to provide an initial IR time point. Reflux at 65° C. is continued for approximately 36 hours. Reflux is discontinued after product confirmation by IR.

### Example 5

Table 2 provides formulations of polymerizable liquids (or hydrogels or build materials) according to some embodiments described herein. In Table 2, “Comp.” means “Composition,” and the amounts listed for a given Composition are weight percents, based on the total weight of the Composition. It is to be understood that all components of a given Composition add up to 100 weight percent. Table 3 provides components of Compositions 1-6. Table 4 provides the Elongation at Break (EOB) for Compositions 1-6, measured as described below in Example 6.

It will be noted that Compositions 1-6 include varying amounts of components described herein (as shown in Table 2). Additionally, Compositions 4-6 in particular use different species of Formula (I), in which the integer n is varied to correspond approximately to weight average molecular weights of 200,600, and 1000 for the PEG portion of Formula (I) (as shown in Table 3). As shown in Table 4, all of Compositions 1-6 have an EOB of 150-300%. Additionally, in Table 3, “QY” refers to quinoline yellow.

TABLE 2
Example Compositions

| Component | Comp. 1 | Comp. 2 | Comp. 3 | Comp. 4 | Comp. 5 | Comp. 6 |
|---|---|---|---|---|---|---|
| Curable Compound | 12 | 20 | 30 | 35 | 35 | 35 |
| (Formula I) | | | | | | |
| Acrylate Component | 39.5 | 40 | 50 | 50 | 50 | 50 |
| Colorant | 0.1 | 0.4 | 0.4 | 0.4 | 0.4 | 0.4 |
| Photoinitiator | 1.4 | 1.8 | 1.8 | 1.8 | 1.8 | 1.8 |
| Water | 47 | 37.8 | 17.8 | 12.8 | 12.8 | 12.8 |

TABLE 3
Components

| Component | Comp. 1 | Comp. 2 | Comp. 3 | Comp. 4 | Comp. 5 | Comp. 6 |
|---|---|---|---|---|---|---|
| Curable | MA- | MA- | MA- | MA- | MA- | MA- |
| Compound | PEG200- | PEG200- | PEG200- | PEG200- | PEG600- | PEG1000- |
| (Formula I) | MA | MA | MA | MA | MA | MA |
| Acrylate | HEAA (N- | HPA + | HPA + | HPA + | HPA + | HPA + |
| Component | hydroxy | HBA | HBA + | HBA + | HBA + | HBA + |
| | ethyl | (hydroxy | HEAA | HEAA | HEAA | HEAA |
| | acrylamide) + | butyl | (15 + | (15 + | (15 + | (15 + |
| | HPA | acrylate) + | 25 + | 25 + | 25 + | 25 + |
| | (hydroxy | HEAA | 10) | 10) | 10) | 10) |
| | propyl | (10 + | | | | |
| | acrylate) | 20 + | | | | |
| | (5 + | 10) | | | | |
| | 34.5) | | | | | |
| Colorant | QY | QY | QY | QY | QY | QY |
| Photoinitiator | Sodium | Sodium | Sodium | Sodium | Sodium | Sodium |
| | TPO-L | TPO-L | TPO-L | TPO-L | TPO-L | TPO-L |

TABLE 4
Elongation at Break

| | Comp. 1 | Comp. 2 | Comp. 3 | Comp. 4 | Comp. 5 | Comp. 6 |
|---|---|---|---|---|---|---|
| Elongation @ | 250 | 167 | 192 | 208 | 188 | 169 |
| Break (%) | | | | | | |

In addition to Compositions 1-6, several comparative compositions were prepared. Specifically, Comparative Compositions 1-3 were prepared in a manner similar to Compositions 1-6. Comparative Compositions 1-3 may be compared to Composition 6 in particular. The components of Comparative Compositions 1-3 were the same as those of Composition 6, except Comparative Compositions 1-3 each completely omit the curable compound of Formula I. The compound of Formula I was replaced in Comparative Composition 1 with water. Thus, Comparative Composition 1 included 47.8 wt. % water rather than 12.8 wt. % water. The curable compound of Formula I was replaced in Comparative Composition 2 with a comparable poly(ethylene glycol) diacrylate. Thus, Comparative Composition 2 included 35 wt. % PEGDA as an alternative curable component. The curable compound of Formula I was replaced in Comparative Composition 3 with a monomeric triacrylate (TAC) that does not include a PEG moiety. Thus, Comparative Composition 3 included 35 wt. % TAC as an alternative curable component.

The EOB was measured for each of Comparative Compositions 1-3, as described in Example 7 below. As shown in Table 5, Comparative Compositions 1-3, which exclude curable compounds of Formula I or Formula II described herein, each had an EOB value below 150%.

TABLE 5
Elongation at Break

| | | Comparative | Comparative | Comparative |
|---|---|---|---|---|
| | Comp. 6 | Comp. 1 | Comp. 2 | Comp. 3 |
| Elongation @ | 169 | 136 | 21 | 23 |
| Break (%) | | | | |

Additional compositions according to the present invention are provided using the amounts in Table 6 below. The amounts in Table 6 refer to the wt. % of each component of the identified composition, based on the total weight of the composition. Additionally, “PI” stands for “photoinitiator.” Moreover, in all cases in Table 6 below, water provides the balance of components to reach 100 wt. % (e.g., 10-60 wt. % water).

TABLE 6
Composition Components

| | Curable | Curable | | Additional | | | |
|---|---|---|---|---|---|---|---|
| | Compound | Compound | Acrylate | Curable | Stabilizer/ | | |
| Composition | (Formula I) | (Formula II) | Component | Component | Inhibitor | Colorant | PI |
| Comp. 7 | 5-40 | 0-10 | 20-40 | 0-10 | 0-1 | 0-2 | 0-5 |
| Comp. 8 | 5-40 | 0-5 | 35-50 | 0-5 | 0.01-1 | 0-2 | 0-5 |
| Comp. 9 | 5-40 | 1-10 | 15-50 | 1-10 | 0.1-2 | 0.1-2 | 0-5 |
| Comp. 10 | 5-25 | 0-15 | 20-40 | 1-15 | 0.01-2 | 0-2 | 0-5 |
| Comp. 11 | 20-30 | 0-10 | 25-45 | 0-5 | 0-2 | 0.1-1 | 0-5 |
| Comp. 12 | 10-30 | 0-10 | 25-40 | 0-5 | 0-0.2 | 0-0.5 | 0-5 |
| Comp. 13 | 10-35 | 0-10 | 30-50 | 0-5 | 0-0.2 | 0-0.5 | 0-5 |
| Comp. 14 | 10-35 | 0-10 | 40-50 | 0-5 | 0-0.2 | 0.1-0.5 | 1-3 |
| Comp. 15 | 15-40 | 0-15 | 15-35 | 0-10 | 0.01-0.1 | 0.1-1.5 | 1-5 |
| Comp. 16 | 10-25 | 1-10 | 15-45 | 0-15 | 0.01-0.5 | 0-2 | 1-5 |
| Comp. 17 | 5-40 | 5-20 | 20-35 | 5-20 | 0.01-1.5 | 0-2 | 0-5 |
| Comp. 18 | 20-40 | 0-10 | 15-35 | 10-20 | 0-1 | 0-1.5 | 1-5 |
| Comp. 19 | 10-30 | 0-5 | 20-45 | 0-10 | 0.05-1.5 | 0.01-1 | 0-5 |
| Comp. 20 | 5-25 | 1-15 | 20-50 | 1-5 | 0.01-2 | 0-0.5 | 1-5 |
| Comp. 21 | 10-30 | 1-5 | 25-40 | 0-15 | 0.01-0.2 | 0.5-1.5 | 1-5 |

### Example 6

Tensile testing of printed articles for determining elongation at break were tested as follows. The test formulation (the ink, build material, or polymerizable liquid) was printed at room temperature (approximately 23-25° C.) into horizontally oriented rings in 20 μm thick layers on a digital light processing (DLP) printer. The rings had a necked region with a defined 1 mm by 1 mm square cross section. The rings were taken off the printer platform and rinsed of uncured material (e.g., by placing the rings in phosphate buffered saline (PBS) or water at room temperature for 10 minutes or less). Then the rings were loaded onto a dynamic mechanical analysis (DMA) system and vertically stretched at 100% strain per minute (at room temperature) until the instrument reached a maximum strain or the sample broke, thereby providing the Elongation at Break (EOB). By finding the slope of the first 10% strain, the modulus is found.

### Example 7

Swelling of printed articles was determined as follows. measured both in its own ink as well as in PBS. Discs with 8 mm diameter and 3 mm thickness were printed with the test formulation (the ink, build material, or polymerizable liquid of interest) and then placed into shallow dishes. Each disc was measured using a Zeiss Microscope to find the printed diameter. In order to measure swelling in phosphate buffered saline (PBS), discs were submerged in Dulbecco's PBS (++). The discs were covered in solution by at least approximately 5 times the volume of the disc. The diameter of the discs was continually measured over the next week at different time points to evaluate shrinkage or swelling or swelling over time. Final swelling was quantified as [Final Diameter (mm)−Initial Diameter (mm)]/Initial Diameter (mm).

Some additional, non-limiting, example embodiments are provided below.

Embodiment 1. A compound having the structure of Formula (I):

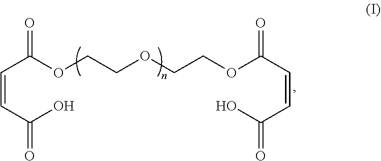

wherein n is an integer between 4 and 40.

Embodiment 2. A compound having the structure of Formula (II):

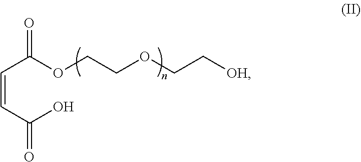

wherein n is an integer between 4 and 40.

Embodiment 3. The compound of Embodiment 1 or Embodiment 2, wherein n is an integer between 4 and 14 or between 4 and 20.

Embodiment 4. The compound of any preceding Embodiment, wherein the compound is a liquid at 25° C. and 1 atm.

Embodiment 5. A hydrogel comprising the compound of any preceding embodiment.

Embodiment 6. The hydrogel of Embodiment 5, wherein the compound is present in the hydrogel in an amount of 10-35 wt. %, based on total weight of the hydrogel.

Embodiment 7. The hydrogel of Embodiment 5 or Embodiment 6 further comprising an acrylate component.

Embodiment 8. The hydrogel of Embodiment 7, wherein the acrylate component is present in an amount of 35-50 wt. %, based on total weight of the hydrogel.

Embodiment 9. The hydrogel of any of Embodiments 5-7, wherein the acrylate component comprises one or more hydroxyalkylacrylates.

Embodiment 10. The hydrogel of any of Embodiments 5-7, wherein the acrylate component comprises one or more polyethylene glycol acrylates or diacrylates.

Embodiment 11. The hydrogel of any of Embodiments 5-7, wherein the acrylate component comprises one or more hydroxyalkylacrylamides.

Embodiment) 2. The hydrogel of any of Embodiments 5-11, wherein water is present in the hydrogel in an amount of 20-85 wt. %, based on total weight of the hydrogel.

Embodiment 13. A polymerizable liquid for hydrogel article formation comprising:
a compound having the structure of Formula (I) or Formula (II):

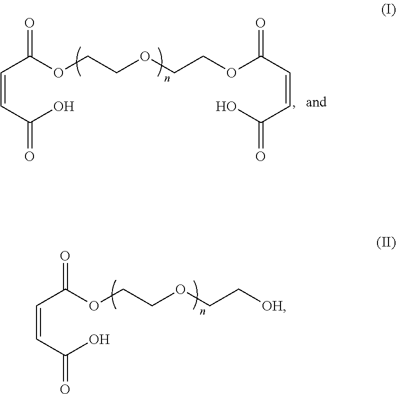

wherein n is an integer between 4 and 40;
an acrylate component; and
water.

Embodiment 14. The polymerizable liquid of Embodiment 13, wherein the polymerizable liquid comprises a compound having the structure of Formula (I).

Embodiment 15. The polymerizable liquid of Embodiment 13 or Embodiment 14, wherein the compound having the structure of Formula (I) or Formula (II) is present in an amount of 10-35 wt. %, based on total weight of the polymerizable liquid.

Embodiment 16. The polymerizable liquid of any of Embodiments 13-15, wherein the acrylate component comprises one or more hydroxyalkylacrylates.

Embodiment 17. The polymerizable liquid of any of Embodiments 13-16, wherein the acrylate component comprises one or more polyethylene glycol acrylates or diacrylates.

Embodiment 18. The polymerizable liquid of any of Embodiments 13-17, wherein the acrylate component comprises one or more hydroxyalkylacrylamides.

Embodiment 19. The polymerizable liquid of any of Embodiments 13-18, wherein the acrylate component is present in an amount of 15-50 wt. % or 35-50 wt. %, based on total weight of the polymerizable liquid.

Embodiment 20. The polymerizable liquid of any of Embodiments 13-19, wherein the water is present in an amount of 20-85 wt. % or 10-50 wt. %, based on total weight of the polymerizable liquid.

Embodiment 21. The polymerizable liquid of any of Embodiments 13-20, wherein the polymerizable liquid further comprises one or more additional polymerizable or curable materials differing from the compound having the structure of Formula (I) or Formula (II) and differing from the acrylate component.

Embodiment 22. The polymerizable liquid of Embodiment 21, wherein the one or more additional polymerizable or curable materials is present in an amount of 1-20 wt. %, based on total weight of the polymerizable liquid.

Embodiment 23. The polymerizable liquid of any of Embodiments 13-22, wherein the polymerizable liquid further comprises one or more colorants.

Embodiment 24. The polymerizable liquid of Embodiment 23, wherein the one or more colorants is present in an amount of 0.1-5 wt. % or 0.1-1 wt. %, based on total weight of the polymerizable liquid.

Embodiment 25. The polymerizable liquid of any of Embodiments 13-24, wherein the polymerizable liquid further comprises a photoinitiator component.

Embodiment 26. The polymerizable liquid of Embodiment 25, wherein the photoinitiator component is present in an amount of 0.1-5 or 0.1-3 wt. %, based on total weight of the polymerizable liquid.

Embodiment 27. The polymerizable liquid of any of Embodiments 13-26, wherein the liquid when polymerized has an elongation at break of greater than 150%, when measured according to the method of Example 6.

Embodiment 28. The polymerizable liquid of any of Embodiments 13-26, wherein the liquid when polymerized has a percent swelling in phosphate buffered saline of less than 20%, when measured according to the method of Example 7.

Embodiment 29. A method of printing a three-dimensional hydrogel article comprising:
providing the polymerizable liquid of any of Embodiments 13-28; and
printing and curing the polymerizable liquid with light to form the hydrogel article.

Embodiment 30. The method of Embodiment 29, wherein the polymerizable liquid is provided in a layer-by-layer process.

Embodiment 31. The method of Embodiment 29 or Embodiment 30, wherein the hydrogel article is a medical implant.

Embodiment 32. A printed three-dimensional article formed from the polymerizable liquid of any of Embodiments 13-28.

All patent documents referred to herein are incorporated by reference in their entireties. Various embodiments of the invention have been described in fulfillment of the various objectives of the invention. It should be recognized that these embodiments are merely illustrative of the principles of the present invention. Numerous modifications and adaptations thereof will be readily apparent to those skilled in the art without departing from the spirit and scope of the invention.

1. A polymerizable liquid for hydrogel article formation comprising:
a compound having the structure of Formula (I) or Formula (II): 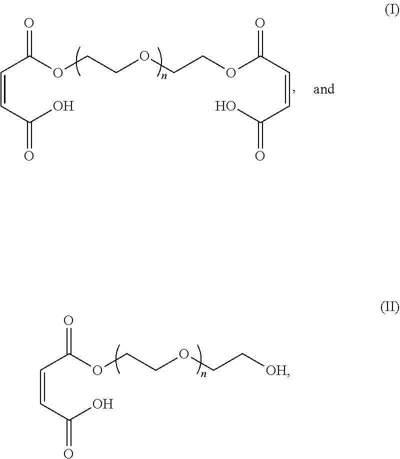
wherein n is an integer between 4 and 40;
an acrylate component; and
water.

2. The polymerizable liquid of claim 1, wherein the polymerizable liquid comprises a compound having the structure of Formula (I).

3. The polymerizable liquid of claim 1, wherein the compound having the structure of Formula (I) or Formula (II) is present in an amount of 4-40 wt. %, based on total weight of the polymerizable liquid.

4. The polymerizable liquid of claim 1, wherein the acrylate component comprises one or more hydroxyalkylacrylates.

5. The polymerizable liquid of claim 1, wherein the acrylate component comprises one or more polyethylene glycol acrylates or diacrylates.

6. The polymerizable liquid of claim 1, wherein the acrylate component comprises one or more hydroxyalkylacrylamides.

7. The polymerizable liquid of claim 1, wherein the acrylate component is present in an amount of 15-50 wt. %, based on total weight of the polymerizable liquid.

8. The polymerizable liquid of claim 1, wherein the water is present in an amount of 20-85 wt. %, based on total weight of the polymerizable liquid.

9. The polymerizable liquid of claim 1, wherein the polymerizable liquid further comprises one or more additional polymerizable or curable materials differing from the compound having the structure of Formula (I) or Formula (II) and differing from the acrylate component.

10. The polymerizable liquid of claim 9, wherein the one or more additional polymerizable or curable materials is present in an amount of 1-20 wt. %, based on total weight of the polymerizable liquid.

11. The polymerizable liquid of claim 1, wherein the polymerizable liquid further comprises one or more colorants.

12. The polymerizable liquid of claim 11, wherein the one or more colorants is present in an amount of 0.1-5 wt. %, based on total weight of the polymerizable liquid.

13. The polymerizable liquid of claim 1, wherein the polymerizable liquid further comprises a photoinitiator component.

14. The polymerizable liquid of claim 13, wherein the photoinitiator component is present in an amount of 0.1-5 weight percent based on total weight of the polymerizable liquid.

15. The polymerizable liquid of claim 1, wherein the liquid when polymerized has an elongation at break of greater than 150%, when measured according to the method of Example 6.

16. The polymerizable liquid of claim 1, wherein the liquid when polymerized has a percent swelling in phosphate buffered saline of less than 20%, when measured according to the method of Example 7.

17. A method of printing a three-dimensional hydrogel article comprising:
providing the polymerizable liquid of claim 1; and
printing and curing the polymerizable liquid with light to form the hydrogel article.

18. The method of claim 17, wherein the polymerizable liquid is provided in a layer-by-layer process.

19. The method of claim 17, wherein the hydrogel article is a medical implant.

20. A printed three-dimensional article formed from the polymerizable liquid of claim 1.
